(19) United States
(12) Patent Application Publication
(10) Pub. No.: US 2004/0009583 A1
(43) Pub. Date: Jan. 15, 2004

(54) Seal for microtiter plate and methods of use thereof

(75) Inventors: James A. Benn, Arlington, MA (US); Douglas R. Smith, Gloucester, MA (US); Joseph D. Michienzi, Mansfield, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genome Therapeutics Corporation

(21) Appl. No.: 10/359,456

(22) Filed: Feb. 5, 2003

Related U.S. Application Data
(60) Provisional application No. 60/354,696, filed on Feb. 5, 2002.

(51) Int. Cl. C12M 1/34

(52) U.S. Cl. 435/287.2

(57) ABSTRACT

A microtiter plate having at least one well and a lid having at least one insert which seals and reduces the volume of the well and methods of using the combination of the well and insert are described herein.

### RELATED APPLICATION

This application claims the benefit of U.S. Provisional Application No. 60/354,696, filed on Feb. 5, 2002.

The entire teachings of the above application are incorporated herein by reference.

### GOVERNMENT SUPPORT

The invention was supported, in whole or in part, by a grant, NHGRI# 1R24 HG 02210. The Government has certain rights in the invention.

### BACKGROUND OF THE INVENTION

One of the most common procedures in recombinant DNA research is PCR (Polymerase Chain Reaction), a standard procedure for amplifying DNA in a sample. PCR comprises a series of denaturing, annealing and extension steps that are repeated for about 30-50 cycles. The denaturing step is typically done at about 94° C.; the annealing step at about 54° C. and the extension step at about 72° C. These three steps, taken together, are often referred to as thermocycling, and the cycles are routinely performed in an automated manner in a thermocycler.

Thermocycling reactions typically use very small sample volumes, for example, microliter (μl) volumes, for often it is difficult to obtain large volumes of sample. One technique is to place microliter samples in a depression, (i.e., a well) in what are called microtiter plates. These plates contain a plurality of wells, typically anywhere from 96 to 384 wells. The samples are also often labeled with a radioactive tag or fluorometric dye. To prevent splashing, spillover, evaporation and condensation of these microliter samples, one procedure is to place a layer of mineral oil over the sample in the well to cover the reaction mixture. However, the oil not only increases the volume of the liquid sample but it can be difficult to remove, thus affecting the accuracy of the results.

Some thermocyclers use heated lids to fit over the sample in the microtiter plates, thus avoiding the use of mineral oil. However, the lids fit loosely on the top of the plate and when the sample temperature rises to near the boiling point some of the sample vaporizes into the air space above the sample. The vapor may also condense on the lid and on the walls of the well above the sample. If enough water vapor were to leave the sample, the concentration of the chemical constituents could increase to the point that the chemical reaction may fail. Thus, it is clear that techniques and/or devices that will permit safe and accurate thermocycling and processing of microliter, or submicroliter, sample volumes is desirable.

### SUMMARY OF THE INVENTION

This invention is related to a processing system capable of processing several microliter and smaller sample volumes, typically 1 microliter or less. The invention significantly reduces safety hazards in processing recombinant DNA samples by reducing the likelihood of splashing and spillover of DNA samples and reactants, especially during thermocycling. Moreover, the use of this invention can result in realizing a 10 fold or more reduction in sequencing reaction costs per sample. In particular, the samples to be thermocycled are frequently being processed for nucleic acid sequencing reactions and contain target DNA that is amplified by polymerase chain reaction (PCR). Thermocycling is integral to PCR. Current thermocycling approaches cannot reliably process volumes less than about 5 microliters and typically use about 10 microliters of sample.

A standard 384 well PCR plate can be used “as-is” to thermocycle volumes as low as 5 microliters. However, when smaller volumes are thermocycled in the same size wells, evaporation and condensation in the wells change the concentrations of the reactants, leading to failure of the PCR reaction. As will be described herein, an insert, also referred to herein as a plug, has been developed to fit into the well of a microtiter plate. The plug described herein effectively fills the unused space of the microtiter well, trapping, for example, a 1 microliter sample in about 2 microliters volume at the bottom of the well, and essentially reducing the volume of the well. In fact, the original volume of the well can be reduced preferably between 50% to 75%, but also 85% or more.

Typically, a sample well from a standard 384 well plate, when loaded with only one microliter of sample has the well volume filled mostly with air. In addition, there are, relatively speaking, large surfaces available on which water vapor from the sample containing hazardous materials (e.g., biological fluids and radioactivity) can condense. In this invention, an insert, or plug, fills the unused space in each well. It seals the bottom of the well, preventing water vapor from escaping from the sample. The insert also prevents sample splashing and spillover, thus significantly reducing sample-to-sample contamination, as well as sample-to-researcher contamination.

A lid has been produced for a standard plate, the lid having an aligned array of plugs (e.g., 384 plugs for a 384 microliter well plate) wherein each plug partially fills one of the wells in the plate and seals it. After thermocycling, the lid is removed, and the sample further processed for a variety of experiments. For example, after thermocycling a 1 microliter sample, 9 microliters of water can be added to the sample, then the diluted sample removed with a multipipettor, easily removing substantially the entire sample for further processing.

As used herein, the term “clean-up” means a process of purification to, for example, separate, isolate or purify, amplified target DNA from contaminating DNA (e.g., unused DNA primer) or after reagents (e.g., chemicals or salts) in a sample.

The thermocycling lid described herein has been tested using standard laboratory protocols, producing sequencing data with similar quality metrics as in standard protocols using much larger volumes. The use of the plugs results in longer, less expensive sequencing reads, and faster, less expensive sample processing times. This system has been used in a semi-production mode to perform single nucleotide polymorphism (SNP) discovery on two genes implicated by cancer. Importantly, the use of this system decreases the safety hazards of recombinant DNA research and the use of biological fluids.

Further encompassed by this invention is a method for processing small volume samples (e.g., less than 10 microliters) when the samples are contained in receptacles (e.g., microtiter plate wells). The test sample comprises target DNA that is to be amplified by PCR. The test sample, typically less than 1 microliter, is placed in the microtiter well with other required reagents so that the total volume of sample in the well is about 1 microliter. A lid mounting the plugs, hereinafter described in greater detail, is placed over the plate and positioned such that each plug is inserted into a well, thus substantially reducing the volume of the well and creating a seal. The sample is then processed, for example, by thermocycling. In a particular embodiment of the present invention, the sample is subjected to ultrafiltration or other methods, such as ethanol (EtOH) methods to clean-up the sample and obtain purified target DNA. The cleaned sample is suitable for use in a sequencing reaction experiment.

Thermocycling small volumes in a standard microtiter plate is challenging due to evaporation loss, which in turn would alter chemical concentrations. The insert/plug described herein substantially reduces the air volume and surface areas in order to inhibit evaporation and condensation of water. The concept of a plug or seal in the form of a reusable insert that reduces air/sample volume, which in some applications is semi-flexible, that can be easily applied and removed, is a vast improvement over older mineral oil techniques and currently used lids. In addition to the thermocycling process, the insert could also be used in steady-state amplification and any other application that benefits the containment of small volume samples. In particular, any process using small volumes that requires holding a constant temperature for several hours is benefited by the present invention.

A seal for microtiter plates for use in performing the above-described method comprises a lid having a plurality of plugs or inserts projecting downwardly from the lid and aligned in an array with each other in the same pattern as the well of a microtiter seal such that each plug enters a designated well of the microtiter plate. Each plug has a sealing portion that can engage with the interior of the well or with a ring around the top of the well to seal it. In order to reduce the effective volume of the well, the plug will occupy a major portion of the volume of the well, so that the volume of the well is reduced at least 50%.

The plug or insert may have various configurations, for example, it may be tapered, conical, hexagonal, square or rectangular. It could be a truncated cone or cylinder. The wells could also be truncated conical configurations or any configuration to engage the plug. Both the plug and well could be cylindrical with the plug of a size to fit into the well. Basically, the lid and the plugs which project downwardly from it are made of a flexible plastic which is normally a chemically inert elastomer. It can also be made of urethane or medical grade silicone rubber or be metal such as stainless steel and coated with a chemically inert elastomer.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a microtiter plate well with a lid above the well and having a plug inserted in the well.

FIG. 2 is a plan view of the well and plug shown in FIG. 1.

FIG. 3 is a detail view of modified versions of the well and plug of FIG. 1.

FIG. 4 shows another embodiment of a microtiter plate well with a lid above the well and having a plug inserted in the well.

FIG. 5 is a plan view of the well and plug shown in FIG. 4.

FIG. 6 is a detailed view of modified versions of the well and plug of FIG. 4.

FIG. 7 is another embodiment of a microtiter plate and well with a lid above the well and having a plug inserted in the well.

FIG. 8 is a plan view of the well and plug shown in FIG. 7.

FIG. 9 is a detailed view of modified versions of the well and plug of FIG. 7.

FIG. 10 shows yet another embodiment of a microtiter plate well with a lid above the well and having a plug inserted in the well.

FIG. 11 is a plan view of the well and plug shown in FIG. 10.

FIG. 12 is a detailed view of modified versions of the well and plug of FIG. 10.

FIG. 13 is a rendering of a lid to a microtiter plate well PCR plate. The cones point up in this view. When inverted, each fit downwardly into a single well of the plate to seal it and reduce its volume.

FIG. 14 is a view of the 384 well thermocycling lid adjacent to a standard 384 well plate.

FIG. 15 shows a capillary electrophoresis electropherogram with results of an experiment thermocycling 1 microliter of sample with UF clean-up.

FIG. 16 shows another embodiment of a PCR plate well with a lid above the well and having a plug inserted in the well.

FIG. 17 shows yet another embodiment of a microtiter plate well with a lid above the well and having a plug inserted in the well.

FIG. 18 shows the gel image results of PCR reactions in microliter sample wells sealed with vinyl lids, aluminum foil lids, and lids with inserts.

### DETAILED DESCRIPTION OF THE INVENTION

A description of preferred embodiments of the invention follows.

The invention described herein relates to a lid having a plug that seals for retaining microliter and submicroliter volumes of samples in the well of a microtiter plate during processing of the samples, especially during thermocycling. The primary cause of poor thermocycling results is evaporation of the sample due to the ratio of sample volume to well volume. Hence the invention is directed to sealing the wells in order to reduce evaporation by reducing the ratio. Condensation on the walls of the well is also a problem. The solution basically resides in inserting into a well, or the wells of a microtiter plate, a plug or other volume consuming insert comprising an annulus shaped to the interior of the well. Another aspect of the invention is to utilize the plug itself in forming a seal. Accordingly, the sample is sealed in the well close to its surface to reduce both evaporation and condensation. The plug seal reduces the volume of air in the well between the sample and the plug seal and also the surface area of the well per se, in order to reduce evaporation and condensation of water and/or other fluids in the well. Also, as a result of the seal, safety concerns for handling biological samples are significantly reduced.

A standard 384 well PCR plate can be used to thermocycle volumes as low as 5 to 10 microliters. However, when smaller volumes are thermocycled, evaporation and condensation in the wells change the concentrations of the reactants leading to failure of the PCR reactants. The lid of the present invention has plugs that fill most of the unused space of each well by trapping one microliter of sample in about 2 microliters or less volume at the bottom of the well. In particular, the inserts (plugs) of the present invention fit one within each well, which effectively fills the unused space in the well and also seals the lower portion of the well preventing water and vapor from escaping from the sample. The inserts and lids of the present invention are suitable for use with any available microtiter plate, and the use of the lids are not limited to amplification reactions. Any process where evaporation, condensation and/or safe handling of liquid samples are of concern will benefit from the present invention.

Essentially, the plug or insert creates a new sealed volume that is smaller than the original well volume. Thus, while many different shapes of plugs or inserts created where the plug does not fill the volume of the well, it will still fulfill the function of sealing the well to make a smaller volume in the actual chamber created that holds the liquid sample to be processed. Therefore, this invention is an insert for a microtiter well for sealing the well and reducing its volume, the insert comprising a plug having an annulus for engaging the well to create a seal with the well, preferably with the interior of the well and reducing more than half of the volume of the well.

A plug of this invention can be made of two or more parts. One part can be a more rigid structure that looks like a conventional microtiter plate lid. The second part can be the sealing portion of the fingers of the lid, which actually make the seal with the sides of the well. This can be an elastomeric material, and can be replicable, intended for single-use, whereas the rigid part is reusable.

The inserts or plugs of the present invention can be made of many different materials. For example, chemically inert elastomeric compounds that will fit against the well surface to form a seal can be used. In particular, the inserts of the present invention can be urethane or medical grade silicone rubber. Alternatively, the inserts or plugs can be metal such as stainless steel or metal plugs coated with a material as described above.

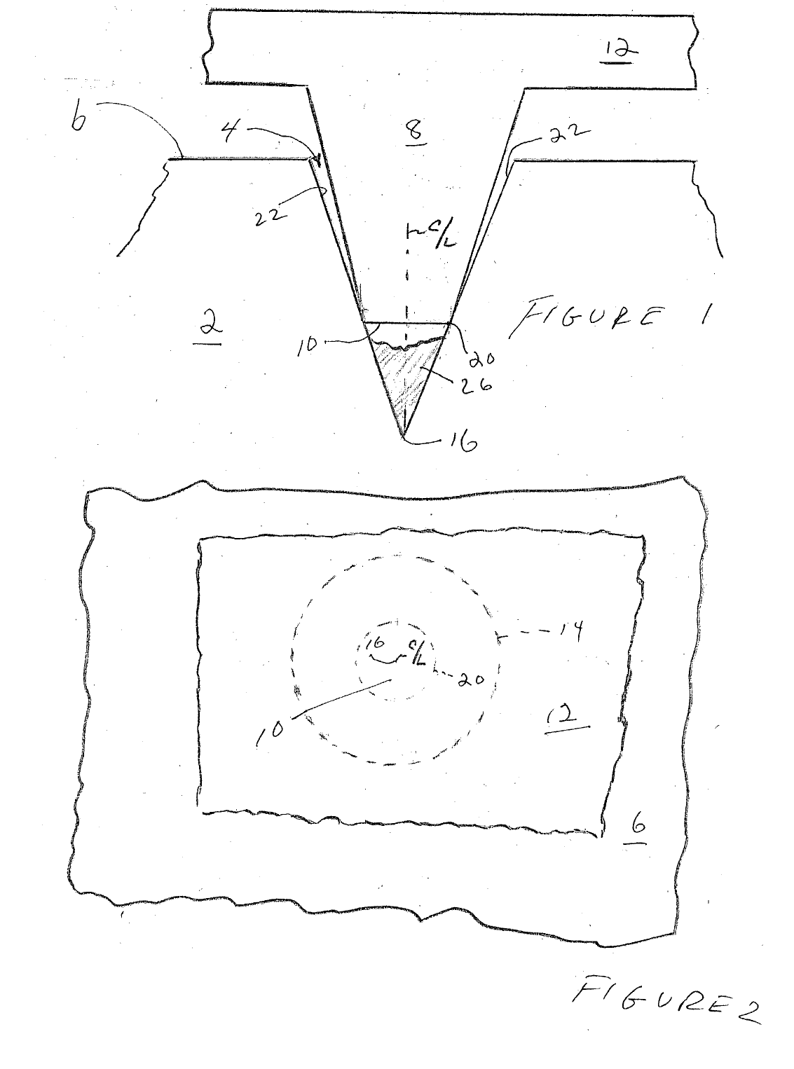

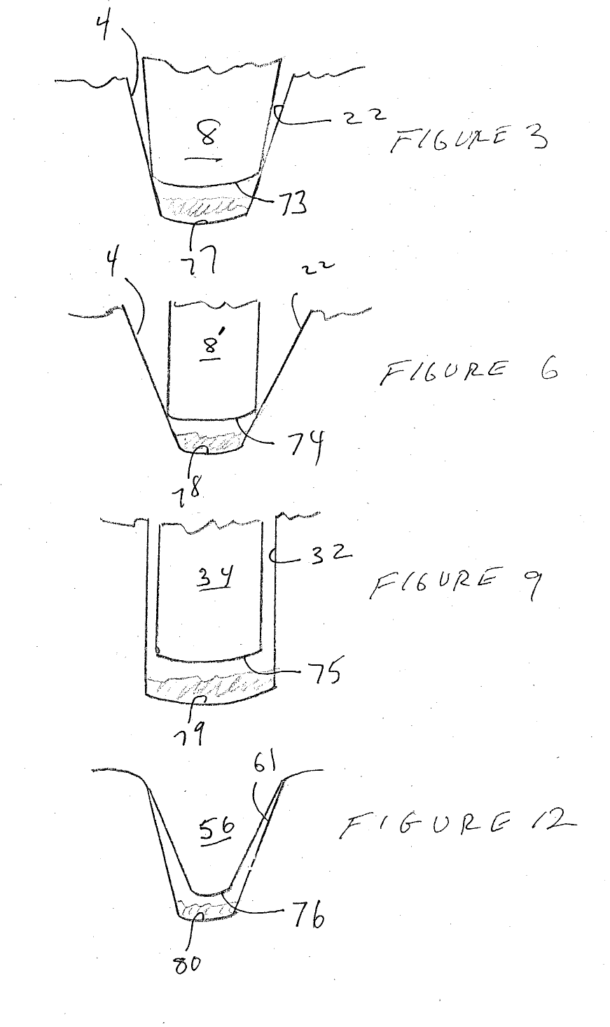

Referring now specifically to FIGS. 1, 2, and 3, a microtiter plate 2 will be seen broken away to show one well 4 of, for example, a 96, or 384 well microtiter plate. The well is in the shape of an inverted cone, having a center line C/L and an included angle of approximately 40 degrees. The horizontal surface of the microtiter plate is designated 6. A well plug 8 is in the form of a truncated cone having a flat bottom 10 at right angles to the center line CL. The plug(s) 8 are formed on the bottom of a lid 12 there being as many plugs as there are wells in the plate 2. The included angle of the plug 8 is less than that of the well 4 being approximately 30 degrees±. The top of the microtiter well 4 is a circle designated 14 (FIG. 2). The bottom or inverted apex of the well being designated 16. The truncated portion 10 of the plug 8 is shaped as a circle designated 20. When the plug(s) are inserted in the well(s), the circular edge annulus 20 of the truncated portion 10 engages the sides 22 of the well.

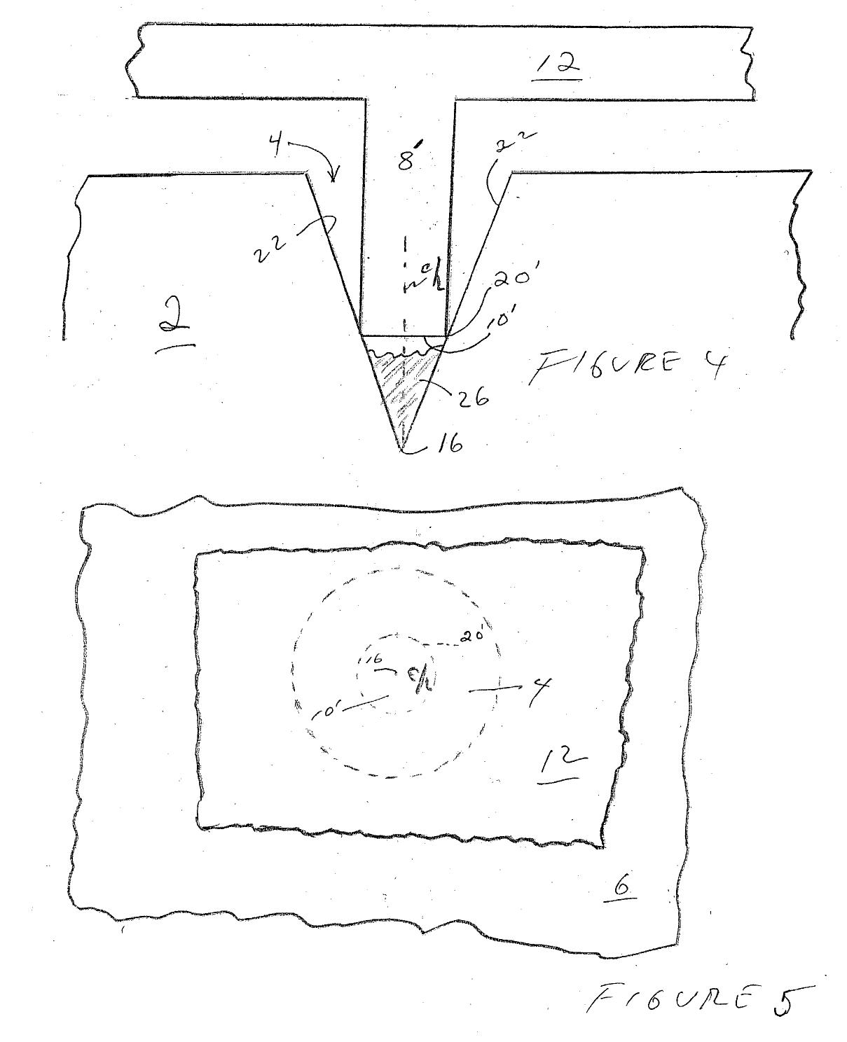

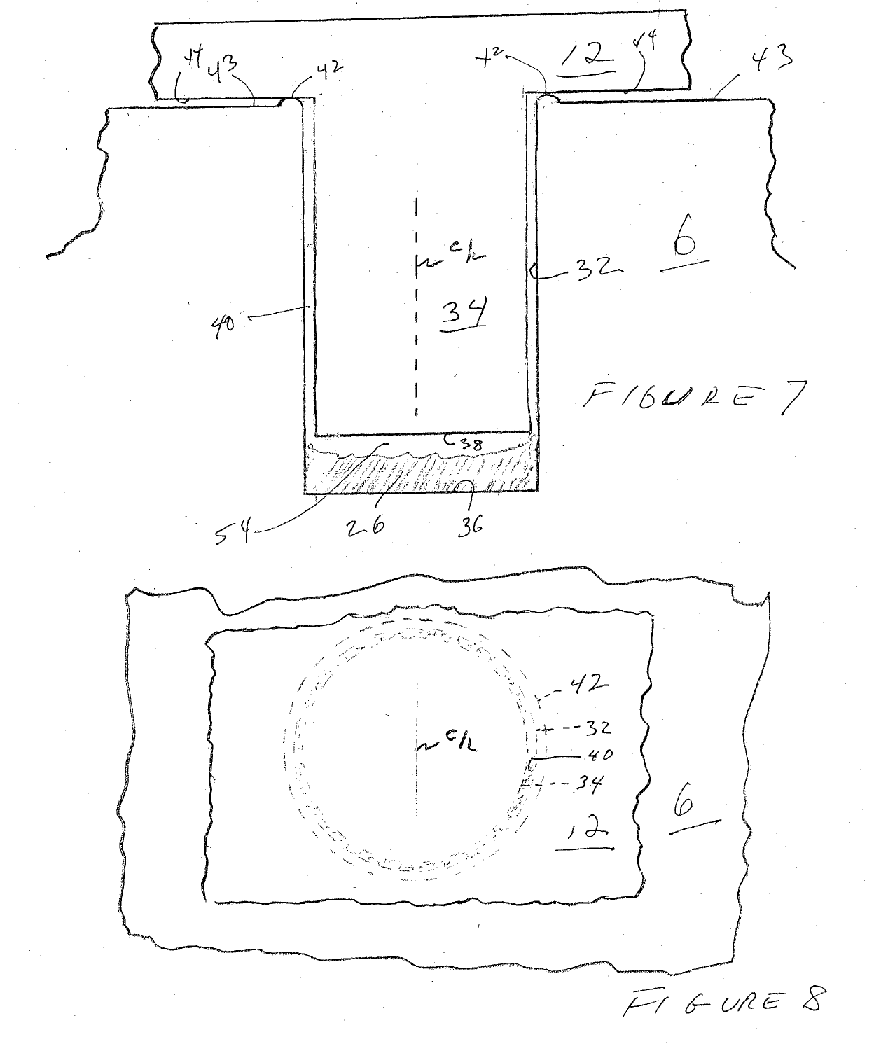

Referring to FIGS. 4 and 6, an alternative embodiment 8′ of the plug 8 will be seen. Whereas the plug 8 shown in FIGS. 1 and 2 is conical, having a flat truncated bottom portion 10, the plug 8′ is cylindrical in cross section. However, it has the same flat circular bottom 10′ as the embodiment shown in FIG. 1, thus making a circular seal 20′ with the walls of the well (S4). In all other respects, the embodiments are the same.

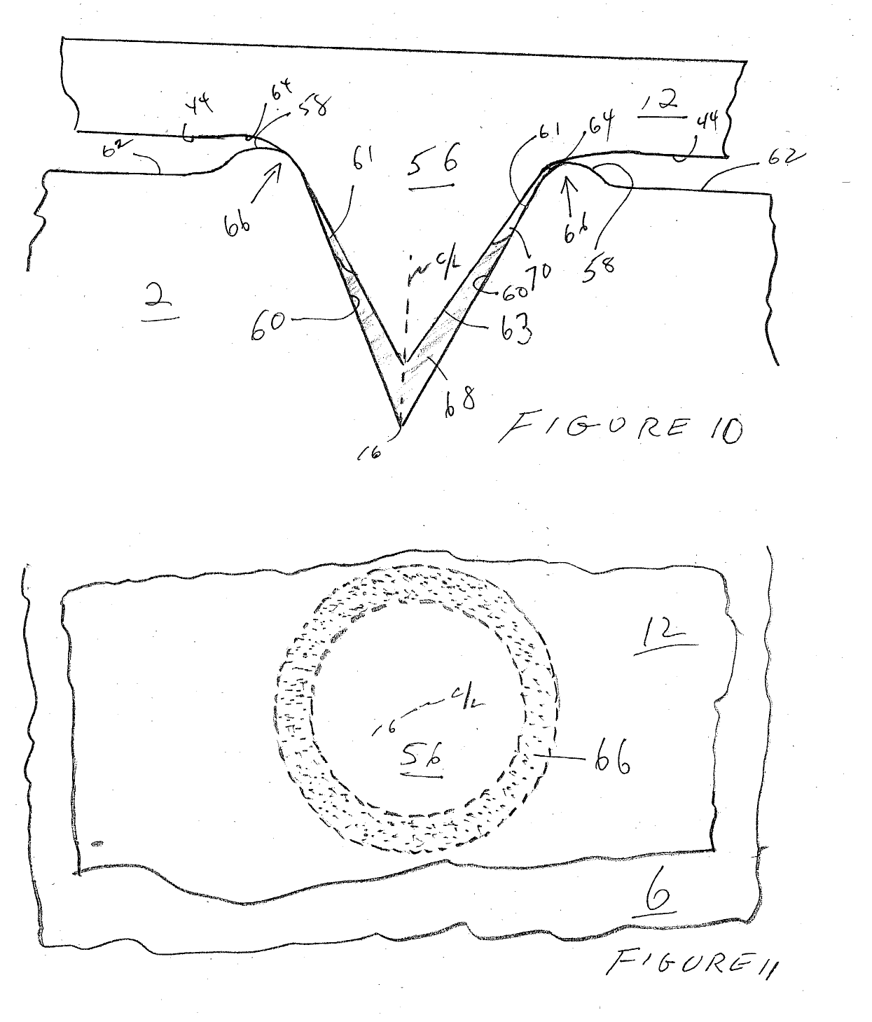

Referring next to FIGS. 7 and 8, another embodiment of the microtiter well 4 and plug 8 will be seen. In this version, the well, designated 32, is cylindrical as is the plug, designated 34. The well has a flat cylindrical bottom 36 and the plug has a flat cylindrical bottom 38. However, in order for the plug 34 to enter the well (both being cylindrical) the plug must have a smaller diameter than of that of the well. Accordingly, a cylindrical space 40 exists between the interior of the well 32 and the exterior of the cylindrical plug 34.

In order to assure sealing of the plug 34 in the well 32, a circular bead 42 is formed on the upper surface 43 of the microtiter plate 6 around the opening of the well 32. When the plug 34 is inserted into the well 32 the bottom 44 of the plate 12 will rest upon the circular bead 42 thus sealing the interior space between the plug and the well. If the volume between the bottom 38 of the plug 34 and the bottom 36 of the well 32 is less than the volume of liquid 26 in the well some of the liquid will rise into the opening 40 between the plug and the well, but will be prevented from leaving the confined area by the seal 42. When the volume between the plug and well is greater than the volume of the liquid 26 in the well an air space 54 will be created between the bottom of the plug and the sample.

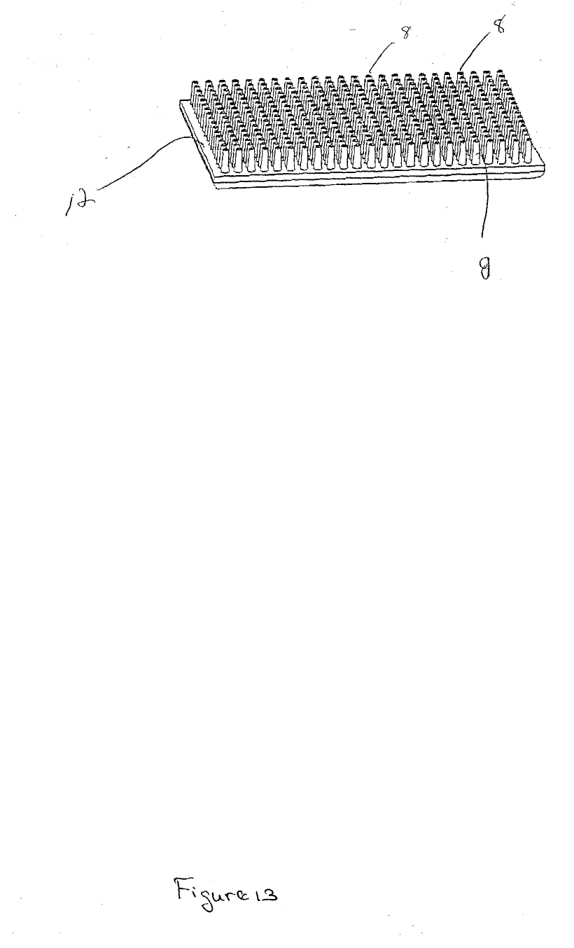

With reference to FIGS. 10 and 11, still another embodiment of the well and plug will be seen. Both the plug, designated 56, and the well, designated 60, are conical. However, the conical angle of the plug 56 is greater than the conical angle of the well 60. Around the periphery of the well 60 is a circular bead 58 smoothly blending with the conical surface 61 of the well and the upper surface 62 of the microtiter plate 2. In like but reverse manner the conical surface 3 of the plug 56 blends as at 64 with the bottom surface 44 of the plate or lid 12. A circular seal is formed in the area 66 where the bead 58 of the well comes in contact with the blend 64. The quantity of liquid 68 initially in the well will control how high it rises in the well between its surface 63 of the plug and the surface 61 of the well. If it is not completely full it leaves an airspace 70.

Referring next to FIGS. 3, 6, 9 and 12, they may be considered as a group, the figures being modifications of FIGS. 1, 5, 7 and 10, respectively. On each of the modifications, the bottoms of the plugs 8, 8′, 34 and 56 are convex as at 73, 74, 75 and 76. Conversely, the bottoms of the wells 77, 78, 79 and 80 may be concave. Functionally, these modifications of the shape of the bottoms of the wells and plugs will be the same but are intended for ease, manufacturing and cleaning.

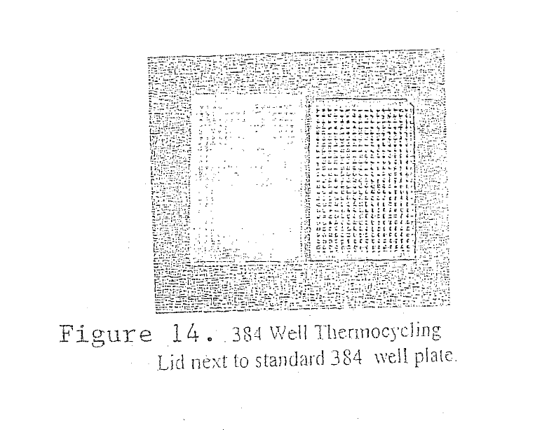

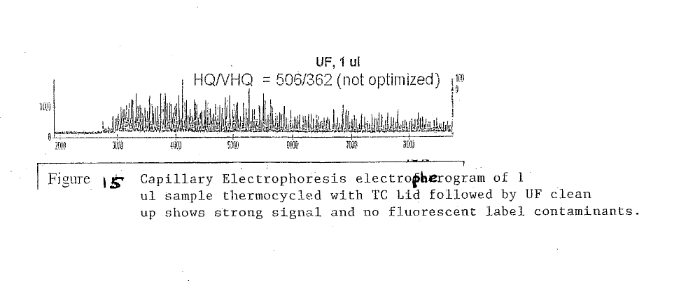

Referring to FIG. 13, there will be seen an inverted lid 12 having a plurality of inserts or plugs 8 in the form of truncated cones. They are aligned in an array with each other to correspond to the array of wells in a microtiter plate (FIG. 14) such that each plug enters a designated well when the lid is urged onto the plate. The thermocycling lid of the present invention fits on standard 384 well PCR plates, results in 2.5 microliter sample volume per well and enables as little as 1 microliter thermocycling in standard thermocyclers.

Referring to FIG. 14, there will be seen an inverted lid 12 next to a standard 384 well plate 81.

Referring to FIG. 15, there will be seen a Capillary Electrophoresis Electropherogram of a 1 μl sample thermocycled with a seal (lid 12 and plugs 8) wherein the sample, following cleanup shows a strong signal and fluorescent label contamination.

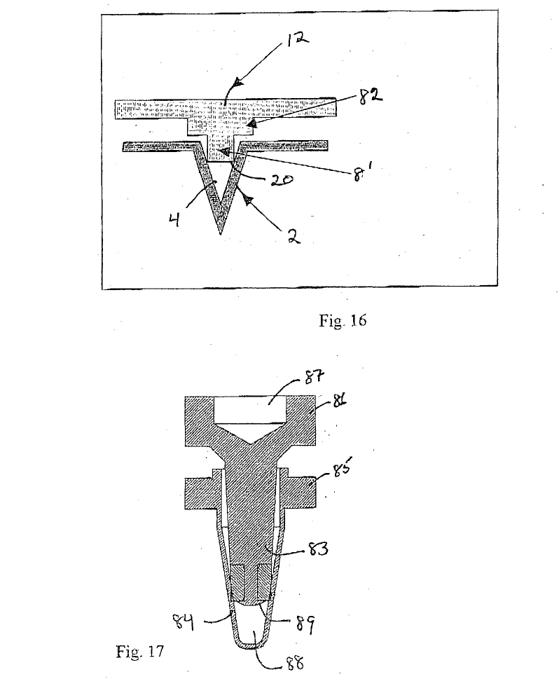

Referring to FIG. 16, there will be seen a microtiter or PCR plate 2 broken away to show one well 4 of a microtiter or PCR plate and the horizontal surface of the plate 6. The plug 8′ is attached to a shoulder 82 between the plug and the lid 12. Both the plug 8′ and the shoulder 12 are cylindrical in cross section. And the plug 8′ has the same flat circular bottom 10″ as the embodiment shown in FIG. 1.

Referring to FIG. 17, there will be seen a microtiter or PCR plate 84 broken away to show one well 88 of a microtiter or PCR plate and the horizontal surface of the plate 85. The rounded truncated portion 89 of plug 83 is shaped as a circle in cross section. The plug 83 is attached to a lid 86 having a top-loading space/modification 87 for insertion of plate/lid movement hardware.

Whereas the inserts or plugs described above and the wells into which they fit have all been described as being circular or acute or cross section. Another embodiment contemplates flat walls as, for example, on a pyramid. The plug could be a polyhedron made up of triangles projecting from a polygonal base which may have many sides or it could be the frustum of polyhedron. In this instance, if the inserts or plugs were this shape, the wells would have to be an inverted frustum having the same number of sides as the plug in order to effect a seal.

A well that can hold 10 μl or more of liquid is desirable to assist in retrieving the small, (i.e, 1 μl or smaller) sample. Since this size sample cannot be captured by standard pipettors, enough more liquid is added to the well to mix with the small sample. Then, standard pipettors are used to retrieve as much of the 10 μl sample as possible. This generally leaves behind from 1 to 2 μl in the well, but captures 80% to 90% of the intended sample.

Automated DNA sequencing is now standard procedure in molecular biology laboratories throughout the world. The initial step of automated sequencing involves amplifying the DNA to be sequenced by PCR using distinctly-labeled nucleotides. As described herein, the use of the inserts or lids of the present invention now allows accurate and safe PCR amplification of very small volumes of sample. After amplification, but before actual determination of the sequence, the PCR mixture must be cleaned-up to eliminate contaminating nucleotides that were not used in the PCR reactions, as well as salts and other chemicals that can affect the accuracy of the sequence determination. Typically, this clean-up step is a precipitation step using chemicals such as ethanol (EtOH) or via an ultrafiltration-based clean-up plate.

A plate that has wells that can hold 10 μl or more is preferred, to assist in retrieving the small 1 μl or smaller samples. Since these cannot be captured by standard pipettors, 10 μl or more is added to the well to mix with the small sample.

Then standard pipettors are used to retrieve as much of the 10 μl sample as possible. This generally leaves behind from 1 to 2 μl in the well, but captures 80% to 90% of the intended sample.

Another advantage of this invention is that the lid can be put on and removed from a microtiter plate by means of a robotic arm thus allowing for faster and more efficient cleanup and removal or storage of samples. For example, suction cups attached to a robotic arm can be lowered onto the lid, and vacuum applied. This will fix the lid to the suction cup, and allow the lid to be lifted from the microtiter plate and placed elsewhere for storage. Traditional lids are held on by, for example, heat-sealing the lid to the top surface of the microtiter plate, or by affixing a sticky-backed tape on the top of the microtiter plate. Both of these traditional lid types can be robotically applied, however their removal must be by manual means, thus preventing automation of the placing and removing of the lids from thermocycling plates.

A further advantage of this invention is that the plug or insert prevents warping of the microtiter plate during thermocycling. When traditional thermocycling lids are used, they have to be heated during thermocycling to prevent condensation of water vapor from the samples, as they are heated to near boiling. This condensation removes water from the PCR reactions and causes them to fail. Therefore, standard peltier block-type thermocyclers have heated bonnets that cover the tops of the microtiter plates and heat the lids on the plates to about 100 deg C., while the bottoms of the microtiter plates are exposed to the different temperature cycles that drive the PCR reaction. This differential heating of the top of the microtiter plates to 100 deg C., while cycling the bottoms of the plates between about 60 deg C. and 96 deg C. causes the plates to become warped. The warped plates are then subsequently hard to use in automated pipetting and plate storage systems, because they jam the mechanisms used to manipulate the plates, and a 96 or 384 well pipettor cannot reach the bottoms of all of the wells of the plate, because the plate will sit unevenly on a platform during pipetting.

The plugs on the thermocycling lid in this invention reach down into the wells of the microtiter plate into the volume of the well that is surrounded by the thermocycler components that heat and cool the plate during thermocycling. Thus the whole volume defined by the microtiter plate and thermocycling lid is heated and cooled at the same temperature during thermocycling. This helps to prevent condensation of water from the sample, and provides a robust PCR reaction. Therefore it is not necessary to heat the microtiter plate lid. When this lid is not heated, the top of the microtiter plate is not heated, preventing extreme differences between the top and the bottom of the microtiter plate and thus preventing warping of the plate. This feature, in combination with the ability to robotically or automatically remove the lid from a microtiter plate after thermocycling, provides an enabling path to downstream automated plate manipulation, storage, pipetting and other protocols using the post-thermocycled microtiter plate.

The sealing lid of this invention can have many features included to be useful to those who want to perform high throughput processing of microtiter plates where this thermocyling lid enables handling of low volumes.

For example, the lid is usable with a wide range of 384 well PCR plates made by different vendors. While the external dimensions of the 384 well PCR plates have very similar dimensions, the internal dimensions (depth and radius) of the wells may vary by 0.25 millimeter or more. These variations in dimensions are sufficient to prevent the lid from adequately sealing if not accounted for in the design of the lid. Designs that allow for these well diameter variations are shown in FIGS. 7, 10, and 16. The lids have a finger that seals each well that is sufficiently long and wide so as to be inserted either more further into the well or less further into the well, to accommodate a larger diameter well or smaller diameter well. The fingers of the lid should be sufficiently long and wide so as to allow sealing of the wells while allowing some clearance space between the lid shoulder and the top of the PCR plate (FIG. 16).

The lid of this invention can be made of a compliant material that promotes good sealing between the insert and the well walls, or the lid can be made of a rigid material, where only the tip of the insert is covered with a compliant material to effect sealing between the insert and the well walls. Thus one can have an insert for a microtiter well for sealing the well and reducing its volume, the insert comprising a plug having an annulus for engaging the well to create a seal with the interior of the well and reducing more than half of the volume of the well wherein the sealing portion the plug is made of a flexible material and the remainder of the insert is made of a more rigid material. For example, the insert can be manufactured wherein the sealing portion of the plug is made of elastomeric sealing media or silicone rubber and the remainder of the insert is made of plastic or metal.

### EXAMPLE 1

### Small Volume Thermocycling

A substantial portion of DNA sequencing cost is due to the sequencing reagents including the fluorescent label. To lower these costs, a novel seal of this invention comprising a lid and plugs were used to thermocycle and sequence a portion of a cancer gene.

In order to baseline systems, multiple subjects were resequenced for a single exon of the BetaCatenin cancer gene, and a consensus sequence was established. Sequences were clipped where base calls differed between subjects. This provided a stringency test for overall base calling accuracy sufficient for single nucleotide polymorphism (SNP) discovery.

Thermocycling volumes of 1 microliter were cleaned up with an ultrafiltration plate. Standard thermocycling and sequencing protocols were used. Results, shown in FIG. 15, produced strong electropherograms with no indications of contaminating fluorescent tags. While no protocol optimization was attempted, read lengths were long enough for most SNP discovery.

Further, 10 microliter thermocycling volumes in a 96 well plate were tested against 2 microliter volumes in a 384 well plate with the novel seal described herein. Results indicate a 10 microliter thermocycling consensus read length of 300 basepairs versus 245 for 2 microliter thermocycling volume. A modified EtOH clean-up was used to minimize DNA losses during clean-up.

### EXAMPLE 2

### Data and Analysis Showing that the Lids with Inserts Allow More Robust PCR than Traditional Microtiter Plate Lids.

The plugs described herein substantially reduce the volume of the microtiter plate sample well. The plug substantially reduces loss of water from the sample during thermocycling, where the temperature of the sample goes from about 55 deg C. to close to about 96 deg C., then back down to 55 deg C. This cycle of heating and cooling may be repeated from 30 to 50 times. For a sample in a sealed sample well, where the sample is brought to 96 deg. C., water evaporates from the sample and enters the air above the sample. This has the result of concentrating the chemical or biological reactants in the sample, leading to sub-optimal or failed reactions. For a sample well volume of 100 microliters, and a sample volume of 1 microliter, the water vapor that will leave the sample and enter the air volume is about 0.06 microliters, causing a concentration of the sample by about 6%. When the sample is then cooled to 55 deg C., then the majority of the vapor in the air condenses on the sides of the sample well rather than back into the liquid sample at the bottom of the well, causing the sample to remain somewhat concentrated. As this heating and cooling is repeated, more and more of the liquid sample volume may be distributed onto the sample well walls in this manner, thus further concentrating the reactants in the sample, until a point is reached where the reaction in the sample fails. When an insert is present in the sample well, the volume available for the water vapor to dissolve into is reduced and also the surface area of sample well available for water to condense on is also reduced. This reduces the amount of water vapor that is removed from the liquid sample, leading to better PCR reactions.

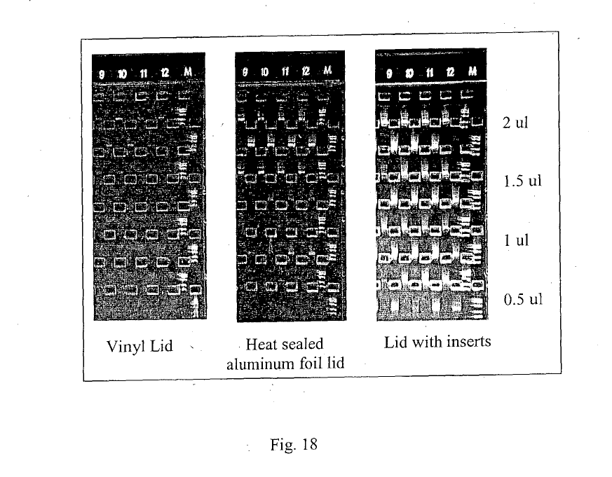

The gel images in FIG. 18 show the results of PCR reactions in 50 microliter sample wells, sealed with a conventional vinyl lid, a conventional heat-sealed aluminum-backed lid, and the novel seal of this invention. Sample volumes of 2.0, 1.5, 1.0 and 0.5 microliters were thermocycled. The results show the amount of DNA amplicon generated by the PCR reactions. All PCR reactions are generally robust with all lids for sample volumes of 5 or more microliters. As can be seen in the images, substantially all PCR reactions from 2.0 down to 0.5 microliters fail when using conventional lids, while these same reactions are successful when using inserts of this invention.

### EXAMPLE 3

### Low Volume Seal Protocols

The thermocycling of sample volumes less than 4 μl in standard 384 well plates generally fails because of water lost from the sample by evaporation. The seals disclosed by this invention prevent this evaporation by effectively reducing the 384 well volumes from 50 μl to about 3 μl. In this example, the seal was designed for use with MARSH 384-well PCR plates (Marsh Bio Products, Inc., Rochester, N.Y.). They are made of urethane, which has been used for ease of prototyping. This material will withstand normal thermocycling temperatures, however, may break down as a result of prolonged or repeated exposure to high temperatures. The seal was allowed to cool prior to handling.

The seal was designed for reuse and is normally cleaned with a detergent. The seal can also be wet autoclaved when the lid is positioned with the plug tips up. Other protocols have been performed by re-aliquoting from 0.5 to 2 μl from a 10 μl sequencing mix. The 10 μl chemistry concentrations are described in Table 1.

TABLE 1

| | | Final Conc. |
|---|---|---|
| Chemistry | 1 Sample | w/DNA |
| RODI (Reverse | 4.7 μl | N/A |
| Osmosis | | |
| Deionized) | | |
| Water | | |
| 5x CSA Buffer | 1.0 μl | 0.5x |
| Primer (10 μM) | 0.32 μl | 0.32 μM |
| Big Dye Mix | 1.0 μl | 0.2 Mix |
| Final Volume | 7.0 μl | 10 μl |
| Template | 3.0 μl | N/A |

The thermocycling protocols are described in Table 2, and steps two through four as illustrated in the Table are generally run for 30 cycles. The heated lid thermocycling option is unnecessary.

TABLE 2

| | Temperature | |
|---|---|---|
| Process | (degrees C) | Time (seconds) |
| Heat Soak | 96 | 60 |
| Denature | 96 | 10 |
| Anneal | 52 | 10 |
| Extension | 68 | 180 |

Alcohol precipitation has been used for post thermocycling clean up.

The sequencing parameters used are described in Table 3.

TABLE 3

| Parameter | Setting |
|---|---|
| Sample Size | 10 μl |
| Injection Time | 10 sec. |
| Voltage | 2 kV |
| Run Time | 2.41 hrs. |

Prior to lid insertion, the reaction samples are in the bottoms of the wells. Then the lid is aligned and inserted into the plate. The lid is symmetrical and can be used in either direction. The lid should be gently pressed down onto the 384 well plate to ensure effective sealing of each well. The thermocyclers bonnet height is adjusted so that a moderate sealing pressure is created and held during cycling. To remove the lid from the plate, firmly grasp the lid and twist clockwise and counterclockwise in small increments while lifting the lid. Once the seal is broken, removal of the lid is easy. The samples can then be retrieved by adding 8-9 μl of buffer or another solution to the wells, then transferred using standard pipetting instruments.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and material are described. Although the invention has been set forth in detail, one skilled in the art will recognize that numerous changes and modifications can be made, and that such changes and modifications may be made without departing from the spirit and scope of the invention.

While this invention has been particularly shown and described with references to preferred embodiments thereof, it will be understood by those skilled in the art that various changes in form and details may be made therein without departing from the scope of the invention encompassed by the appended claims.

What is claimed is:

1. An insert for a microtiter well for sealing the well and reducing its volume, the insert comprising a plug having an annulus for engaging the well to create a seal with the interior of the well and reducing more than half of the volume of the well.

2. An insert according to claim 1 wherein the plug is tapered.

3. An insert according to claim 1 wherein the plug is a cone.

4. An insert according to claim 1 wherein the plug is a truncated cone.

5. An insert according to claim 1 wherein the plug is cylindrical.

6. An insert according to claim 1 wherein the plug is a pyramid.

7. An insert according to claim 1 wherein the plug is a polyhedron, square or rectangular.

8. An insert according to claim 1 wherein the plug is truncated polyhedron, truncated square or truncated rectangular.

9. An insert according to claim 1 wherein the plug is a flexible plastic.

10. An insert according to claim 1 wherein the plug is made of a chemically inert elastomer compound.

11. An insert according to claim 1 wherein the plug is made of urethane.

12. An insert according to claim 1 wherein the plug is made of silicone rubber or medical grade silicone rubber.

13. An insert according to claim 1 wherein the plug is made of metal.

14. An insert according to claim 1 wherein the plug is made of stainless steel.

15. An insert according to claim 1 wherein the plug is made of metal and coated with a chemically inert elastomer.

16. An insert for a tapered microtiter well for sealing the well and reducing its volume comprising a plug having an annular sealing portion engagable with the tapered interior of the well while the plug occupies more than half of the volume of the well.

17. A microtiter well according to claim 16 wherein the well is conical.

18. A microtiter well according to claim 16 wherein the plug is cylindrical.

19. An insert for a microtiter well according to claim 16 wherein the insert is tapered.

20. An insert for a microtiter well according to claim 16 wherein the insert is a truncated cone.

21. An insert for a microtiter well according to claim 16 wherein the insert is a pyramid.

22. An insert for a microtiter well according to claim 16 wherein the insert is a polyhedron.

23. An insert for a microtiter well according to claim 16 wherein the insert is a truncated polyhedron.

24. An insert for a microtiter well for sealing the well and reducing its volume comprising a plug having a sealing portion engagable with the interior of the well while occupying a major portion of its volume wherein both the well and the insert are tapered.

25. An insert for a microtiter well for sealing the well and reducing its volume comprising a plug having a sealing portion engagable with the interior of the well while the plug occupies a major portion of the volume of the well wherein both the well and the insert are tapered and the angle of taper of the plug is greater than that of the well.

26. An insert for a microtiter well for sealing the well and reducing its volume comprising a plug having a sealing portion engagable with the interior of the well while the plug occupies a major portion of the volume of the well wherein the well is tapered and the plug is cylindrical.

27. An insert according to claim 1 wherein the bottom of the insert is convex.

28. An insert according to claim 1 wherein the bottom of the well is concave.

29. An insert according to claim 1 wherein the bottom of the plug is convex and the bottom of this well is concave.

30. The combination of a microtiter plate with at least one well in the plate and a plug having a sealing portion engagable with the interior of the well for sealing the well while the plug occupies a major portion of the volume of the well.

31. The combination of claim 30 wherein the well is tapered.

32. The combination of claim 30 wherein the plug is tapered.

33. The combination of claim 30 wherein both the well and the plug are tapered.

34. The combination of claim 30 wherein the well is cylindrical.

35. The combination of claim 30 wherein the plug is cylindrical.

36. The combination of a microtiter plate with at least one well in the plate and a plug for sealing the well, a circular bead surrounding the well and engagable with the plate when the plug is inserted into the well.

37. The combination of claim 36 wherein the plug is tapered.

38. The combination of claim 36 wherein the well is tapered.

39. The combination of claim 36 wherein both the well and the plug are tapered.

40. The combination of claim 36 wherein the plug is cylindrical.

41. The combination of claim 36 wherein the well is cylindrical.

42. The combination of claim 36 wherein both the plug and the well are cylindrical.

43. The combination of claim 36 wherein both the plug and the well are polyhedrons.

44. The combination of a microtiter plate and a lid, the plate having at least one well and the lid having at least one plug shaped to fit into the well, the well being formed in a surface of the plate and the plug projecting from a surface of the lid and having a sealing portion engagable with the interior of the well while the plug occupies at least one half of the volume of the well such that when the lid is superposed on the plate, the plug enters the well to seal the well and reduce its volume.

45. The combination of claim 44 wherein there are a plurality of wells in the plate and a plurality of plugs projecting from the lid.

46. The combination of claim 44 wherein the wells and the plugs are in aligned array with each other when the lid is on the plate such that each plug enters a designated well when the lid is urged onto the plate.

47. An insert for a microtiter well for sealing the well and reducing its volume comprising a plug insertable in the well and having means for sealing the well and being of a size sufficient to occupy more than half of the volume of the well.

48. A method for thermocycling a small volume sample contained in a microtiter well, comprising the steps of:
a.) placing an insert in the well to reduce the well volume to 50% or more and seal the well; and
b.) thermocycling the sample.

49. A method for processing a small volume sample contained in a microtiter well to obtain target DNA present in the sample, comprising the steps of:
a.) placing an insert in the well to reduce the well volume 50% or more and seal the well;
b.) thermocycling the sample; and
c.) passing the thermocycled sample through a planar ultrafiltration membrane to obtain target DNA.

50. The method of claim 42 wherein the volume of sample is about 1 microliter.

51. A method of processing a small volume sample in microtiter well wherein the processing comprises thermocycling, the method comprising thermocycling the sample

52. An insert for a microtiter well for sealing the well and reducing its volume, the insert comprising a plug having an annulus for engaging the well to create a seal with the interior of the well and reducing more than half of the volume of the well wherein the sealing portion the plug is made of a flexible material and the remainder of the insert is made of a more rigid material.

53. The insert of claim 52 wherein the sealing portion of the plug is made of elastomeric sealing media and the remainder of the insert is made of plastic.
